SEQUENCE LISTING (13 sequences)

SEQ ID NO: 1 - DNA, 51 bp, Artificial Sequence
  DNA encoding an sh RNA
ggagatcacactgacctggcatttgtgtagtgccaggtcagtgtgatctcc

SEQ ID NO: 2 - DNA, 51 bp, Artificial Sequence
  DNA encoding an shRNA
ggagatcacgtgtacctggcatttgtgtagtgccaggtacacgtgatctcc

SEQ ID NO: 3 - DNA, 51 bp, Artificial Sequence
  DNA encoding an sh RNA
cacctgccatgtgcagcatgatttgtgtagtcatgctgcacatggcaggtg

SEQ ID NO: 4 - protein, 9 aa, HIV
RLRPGGKKK

SEQ ID NO: 5 - DNA, 20 bp, Artificial Sequence
  Primer
gctgtggtggtgccttctgg

SEQ ID NO: 6 - DNA, 20 bp, Artificial Sequence
  Primer
cctgggcactgtcactgctt

SEQ ID NO: 7 - RNA, 57 nt, Artificial Sequence
  sh RNA
ggagaucacacugaccuggcauuuguguagugccaggucagugugaucuccuuuuuu

SEQ ID NO: 8 - RNA, 57 nt, Artificial Sequence
  shRNA
ggagaucacguguaccuggcauuuguguagugccagguacacgugaucuccuuuuuu

SEQ ID NO: 9 - RNA, 57 nt, Artificial Sequence
  shRNA
caccugccaugugcagcaugauuuguguagucaugcugcacauggcagguguuuuuu

SEQ ID NO: 10 - DNA, 23 bp, Artificial Sequence
  siRNA
cacacugaccuggcagcgggatg

SEQ ID NO: 11 - DNA, 23 bp, Artificial Sequence
  siRNA
aacgggaaggagacgcugcagtt

SEQ ID NO: 12 - DNA, 23 bp, Artificial Sequence
  siRNA
cacagacucaccgaguggacctg

SEQ ID NO: 13 - DNA, 23 bp, Artificial Sequence
  siRNA
caccugccaugugcagcaugatt

(19) United States
(12) Patent Application Publication
(10) Pub. No.: US 2005/0227940 A1
(43) Pub. Date: Oct. 13, 2005

(54) Amplifying interfering RNA (RNAi) expression and effects

(75) Inventors: John Rossi, Alta Loma, CA (US); Daniela Castanotto, Altadena, CA (US); Laurence Cooper, Sierra Madre, CA (US); Sergio Gonzalez, Duarte, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 11/040,098

(22) Filed: Jan. 24, 2005

Related U.S. Application Data
(60) Provisional application No. 60/538,229, filed on Jan. 23, 2004.

(51) Int. Cl. A61K 48/00

(52) U.S. Cl. 514/44

(57) ABSTRACT

Methods for amplifying expression of interfering RNA (RNAi), and preferably siRNA or shRNA, using a RNAi (si/shRNA)-expressing concatamer, are disclosed. The methods are useful for modulating, including down-regulating and/or inhibiting, expression of a target gene in cells, including mammalian cells.

This application claims priority to U.S. Provisional Application No. 60/538,229, filed Jan. 23, 2004, the entire contents of which are incorporated herein by reference.

### STATEMENT REGARDING FEDERALLY SPONSORED RESEARCH

This invention was made in part with Government support in the form of Grant No. NC1 PO1 CA30206, from the United States Department of Health and Human Services, National Cancer Institute. The United States Government may have certain rights in this invention.

### FIELD OF THE INVENTION

The present invention relates to interfering RNA (RNAi). In particular, the invention relates to approaches for improving RNAi, including small interfering RNA (siRNA). The present invention also relates to reducing expression of major histocompatibility complex (MHC) molecules in mammalian cells using RNAi, preferably using the approaches described herein for improving RNAi.

### BACKGROUND OF THE INVENTION

Post-transcriptional suppression of targeted endogenous gene expression in mammalian cells can be achieved by introduction of sequence-specific small synthetic siRNA duplexes (Elbashir, S. M., et al., 2001; Harborth, J., et al., 2001), and by de novo intracellular synthesis of short sequence-specific double stranded (ds) RNAs, including siRNAs (Yu, J. Y. et al., 2002), which typically contain about 21 to about 25 base pairs. Several groups have demonstrated that siRNAs can be effectively transcribed by Pol III promoters in human cells and elicit target specific mRNA degradation. (Lee, N. S., et al., 2002; Miyagishi, M., et al., 2002; Paul, C. P., et al., 2002; Brummelkamp, T. R., et al., 2002; Ketting, R. F., et al., 2001). Also, siRNAs directed to gene promoter regions as opposed to mRNA have been shown to suppress gene expression by transcriptional gene silencing (TGS) (Morris, K. V., et al., 2004; Kawaski, H., et al., 2004). The above siRNAs, and methods for using them, are the subject of co-pending application Ser. No. 10/776,635, filed Feb. 12, 2004, and entitled “A Method for Directing DNA Methylation Using Homologous, Short Double Stranded RNAs,” which is based on U.S. Provisional Application No. 60/447,013, filed Feb. 14, 2003, both of which are incorporated herein by reference.

siRNA is reported to have been originally discovered in Caenorhabditis elegans by Fire and Mello. RNA interference (RNAi) encompasses a suite of homology-dependent gene silencing mechanisms that are triggered by double-stranded RNA (dsRNA). RNAi is an evolutionarily conserved response, and mechanistically related processes exist in plants, animals, and fungi. RNAi is a phenomenon in which a dsRNA specifically suppresses the expression of a gene bearing its complementary sequence. Current evidence suggests that RNA interference and other “RNA silencing” phenomena reflect an elaborate cellular apparatus that eliminates abundant but defective messenger RNAs and defends against molecular parasites such as transposons and viruses. RNAi is a flexible gene silencing mechanism that responds to double-stranded RNA by suppressing homologous genes. The application of RNAi in cultured mammalian cells is also well known. Elbashir et al. designed 21-nucleotide siRNA duplexes, with symmetric two-nucleotide 3′ overhangs that were transfected into mammalian cells without inducing the antiviral response. The siRNA duplexes reduced gene expression in a cell-type-specific manner. Silencing of endogenous genes has been demonstrated using siRNAs. RNAi techniques also may be more efficient than current methods, such as antisense RNA.

Pol III RNA promoters (Paule, M. R., et al., 2000), such as the U6 small nuclear RNA promoter, can be used to stably express siRNA in mammalian cells (Miyagishi, M., et al., 2002; Lee, N. S., et al., 2002; Paul, C. P., et al. 2002). However, the interfering effect of the expressed siRNA is sometimes or often insufficient to achieve a desired phenotype. One mechanism to improve efficacy is to screen candidate siRNAs for optimal activity since positional effects can alter the extent of inhibition (Holen, T., et al., 2002; Sohail, M., et al., 2003). However, accessible siRNA target sites may be rare in some human mRNAs and the relative effectiveness of the expressed siRNA may be difficult to predict. Achieving desired levels of knockdown is a barrier to successful analytic and therapeutic application.

The major histocompatibility complex (MHC) class I genes in human T cells are a large group of closely linked genes on chromosome 6 encoding the classical human leukocyte antigen (HLA) ABC molecules, that together with b₂-microglobulin are expressed on nearly all nucleated human cells.

HLA are glycoproteins and consist of a highly polymorphic, heavy chain (˜45 kDa) associated noncovalently with b₂-microglobulin (˜12 kDa) The heavy chain is composed of three external domains designated a1, a2, and a3, a single transmembrane segment, and a cytoplasmic tail of 30 to 40 residues.

Pulse-chase biosynthetic experiments have indicated that the association of heavy chain and b₂m protein occurs rapidly after translation, presumably in the ER. Studies with the human Daudi cell line, which has a mutant b₂m gene and thus lacks b₂m protein, have indicated that b₂m is generally required for expression of HLA class I molecules at the cell surface. The classical HLA class I genes expressed by Jurkat cells are known and are HLA-A*0301 (homozygous), HLA-B*0702, HLA-B*3503, HLA-C*0401, and HLA-C*0702.

The MHC class I molecules are present at the surface of virtually all nucleated healthy cells where they play an essential role in the presentation of viral or turnover-associated Ag to cytotoxic T cells (CTL). The classical HLA class I molecules function both as alloantigens to trigger immune recognition and graft rejection of allogeneic cells in unmatched recipients (Parham, P., 1999) and as a platform to present self or foreign peptides that can be recognized by CD8⁺ T cells bearing a clonotypic T-cell receptor. (Adams et al., 2001.)

The ability to down-regulate, eliminate and/or change MHC expression may allow cells to escape from cellular immune surveillance. This has important implications for the fields of:

1.) Transplantation

The success of hematopoietic stem-cell transplantation (HSCT) and solid-organ transplantation depends on preventing rejection of the graft by the endogenous immune response. In the case of HSCT, care must also be taken to avoid creating an immune response between the introduced immune cells and normal host tissues. These two problems, graft rejection and graft-versus-host-disease (GVHD), can be overcome by matching the MHC molecules between donor and host in a process known as HLA typing and by using immunosuppressive medications to limit the activity of the immune response. The dependence of the transplant field on HLA typing and immunosuppression has major implications. (A) It limits the number of acceptable donor-recipient pairs leading to an inequality between excess demand and limited supply. (B) The immunosuppressive medications cause significant morbidity and mortality due to the emergence of opportunistic infections. Altering the MHC expression of the graft to avoid allo-responses will improve these limitations.

2. Gene Therapy

A major obstacle to the in vivo persistence of cells modified by vectors is the development of a host immune response to transgene or vector-encoded proteins. Studies in which gene-modified cells have been inoculated into immunocompetent animals and humans have shown that potent host immune responses to transgene encoded proteins such as neomycin phosphotransferase, hygromycin phosphotransferase, herpes simplex virus thymidine kinase, and therapeutic genes limits the in vivo persistence of transferred cells. The immune mechanisms responsible for eliminating genetically altered cells include antibody responses to transgene products that were secreted or expressed at the cell surface and cytotoxic T-cell responses to peptide fragments derived from intracellular proteins. Since the introduced genes result in proteins that are presented via MHC molecules, altering expression of MHC molecules may decrease the ability of the recipient's immune response to respond to the introduced genes.

Altering the MHC expression of the graft to avoid the cellular immune response to introduced transgenes will improve the survival of genetically modified cells in vivo.

3. Immunology

T cells use the αβ TCR to respond to cognate antigen in the context of MHC class I molecules. Thus, APCs used in vitro or in vivo must share HLA molecules. To avoid an allo-response directed against non-shared HLA molecules, investigators usually use autologous antigen presenting cells (APCs) for antigen stimulation in vitro and vaccination in vivo.

Reducing the expression of non-shared MHC molecules will avoid the generation of an allo-immune response and simultaneous co-expression of desired MHC molecules will allow APCs to be developed from a common donor pool that will facilitate the development of vaccines.

What is needed is an approach for improving the effectiveness of siRNA technology. In particular, a solution is needed to the problem that in many cases accessible siRNA target sites may be rare in some human mRNAs and that the relative effectiveness of the expressed siRNA may be difficult to predict. More specifically, an approach is needed to achieve the desired levels of knockdown for successful applications of RNAi.

### SUMMARY OF THE INVENTION

In the present invention, the effects of interfering RNA (RNAi), preferably small interfering RNA (siRNA) or short hairpin RNA (shRNA), which is a form of siRNA and functionally operates like siRNA, can be enhanced by amplifying RNAi expression. The approach of the present invention is useful for achieving maximal target down regulation, even when the choice of optimal siRNA-binding sites is limited.

In one aspect, the invention relates to a method for amplifying expression of double-stranded RNA, preferably RNAi, in a cell, preferably a mammalian cell. The method comprises generally introducing a plurality of expression cassettes encoding double-stranded RNA, including siRNA or shRNA. The method preferably comprises introducing the expression vehicles into the cell together as a single unit, and more preferably as a RNAi-expressing concatamer, preferably a siRNA- or shRNA-expressing (collectively si/shRNA) concatamer, which is more preferably in the form of an expression vector, comprising a plurality of promoter-RNAi (si/shRNA) expression cassettes, one or more of which express RNAi (si/shRNA).

In another aspect, the invention relates to a method for down-regulating and/or inhibiting expression of a target gene in a cell such as a mammalian cell. The method preferably comprises introducing into the cell a RNAi (si/shRNA)-expressing concatamer, more preferably in the form of an expression vector, comprising a plurality of promoter-RNAi (si/shRNA) expression cassettes, wherein RNAi (si/shRNA) is expressed and initiates RNA interference of expression of the target gene, thereby down-regulating and/or inhibiting expression of the target gene. The method of the present invention is useful for any target, including those for which higher levels of RNAi expression are required to effect RNAi.

The target gene can be any gene, and in one embodiment is a gene associated with the immune system, preferably a gene encoding MHC (e.g., MHC class I or II) and more preferably a MHC class I gene.

In another aspect, the present invention relates to reducing expression of MHC molecules, or other components of antigen processing, in cells, including mammalian cells, using RNAi, preferably siRNA or shRNA.

In another aspect, the invention relates to a RNAi-expression concatamer, preferably a siRNA-expression concatamer or shRNA-expression concatamer, and also preferably in the form of an expression vector, comprising a plurality of promoter-si/shRNA expression cassettes.

The present invention can be used to express multiple RNAi (si/shRNA) against different target sites within the same gene, or against target sites contained in two or more different genes.

In another aspect, the invention relates to methods for making a RNAi-expression concatamer according to the invention, preferably using selected restriction enzymes or other workable means known in the art for constructing such a concatamer.

The present invention thus is useful in the design and production of expression vectors to achieve desired levels of gene modulation or inhibition by expressed RNAi (si/shRNA).

The dose-dependent effect made possible by the present invention is accomplished preferably using multiple copies of RNAi (si/shRNA), preferably placed under control of a Pol III RNA promoter, more preferably a U6 small nuclear (Pol III) RNA promoter.

In one embodiment, the relative down-regulation of expression of HLA genes, preferably classical HLA class I genes, is titrated in T cells, or other cells, by varying the number of promoter and stem-loop cassettes in order to modulate the level of expression of RNAi (si/shRNA) specific for the HLA genes.

In one application, interfering with expression of MHC class I genes using siRNA homologous with a sequence conserved in most classical polymorphic HLA-A, -B and -C loci offers a mechanism to help prevent rejection of an allogeneic graft or cells that express immunogenic vector-encoded transgenes. In particular, a dose-dependent RNAi-effect was accomplished placing copies of shRNA under control of the Pol III U6 small nuclear RNA promoter in tandem in a DNA vector. Using this system, simultaneous down-regulation of expression of classical human leukocyte antigen (HLA) class I genes was achieved in cultured and primary human T cells.

The system of the present invention offers numerous applications and advantages, including being useful to help circumvent T cell-mediated rejection of immunogenic and/or HLA-disparate allografts.

### BRIEF DESCRIPTION OF FIGURES

The si/shRNA expression vector targeting HLA contains multiple cassettes under the control of a U6 Pol III promoter expressing small-hairpin RNAs specific for the HLA target.

FIG. 1A shows a diagram of an expression cassette containing a U6 Pol III promoter and one copy of the 705 stem-loop cassette. (The designation SLAS stands for Stem Loop Anti-Sense and depicts the orientation in which the stem-loop sequence was constructed). The “705” designation refers to a nucleotide sequence complimentary with human HLA mRNA.

FIG. 1B shows a diagram of a U6 promoter and HLA ABC-specific, scrambled, or HLA A-specific hairpin-loop cassette (not to scale). The 9 nucleotide hairpin loops and 6 nucleotide terminator sequences are shown in lower case. The 375 bp human U6 Pol III promoter (−264 to +1) and small hairpin (sh) RNA cassettes were constructed by PCR (Castanotto et al., 2002) with inclusion of a Sal I restriction enzyme (RE) site 5′ to the promoter and insertion of the shRNA sequences at position +1 of the U6 transcripts. The shRNA sequences are followed by the Pol III terminator signal (TTTTTT) and two RE sites (XhoI and Not I) to form a U6shRNA cassette. The cloned U6shRNA cassettes were validated by DNA sequencing. The DNA encoding the HLA ABC-specific shRNA sequence was 5′-GGAGATCACACTGACCTGGCAtttgtgtagTGCCAGGTCAGTGTGATCTCC-3′ [SEQ ID NO: 1]. The DNA encoding the scrambled variant of this shRNA sequence was 5′-GGAGATCACGTGTACCTGGCAtttgtgtagTGCCAGGTACACGTGATCTCC-3′ [SEQ ID NO. 2]. The DNA encoding the HLA A-specific shRNA sequence was 5′-CACCTGCCATGTGCAGCATGAtttgtgtagTCATGCTGCACATGGCAGGTG-3′ [SEQ ID NO. 3]. To accommodate the requirement for an initiating G in U6 transcripts, needed for efficient priming of transcription, an additional guanine was included prior to the first 5′ nucleotide of the HLA A-specific shRNA sequence. (Lee, N. S. et al., 2002) The hairpin-loop (tttgtgtag) is shown as lower-case lettering. One to eight copies of the U6 promoter and dsRNA (designated U6shRNA) cassettes, digested with Sal I and Not I were directionally cloned into the unique Xho I and Not I sites of the EGFP/Neo-diipMG or HyTK-pMG plasmids, and the number of inserted copies was validated by sequencing and by agarose gel electrophoresis after digesting with Not I and Eco RI (HLA ABC-specific stem-loop: [SEQ ID NO: 7], scrambled stem-loop: [SEQ ID NO: 8], HLA A-specific stem-loop: [SEQ ID NO: 9]).

FIG. 1C shows a schematic of a DNA plasmid backbone designated EGFP/diipMGNeo, which does not contain the U6 promoter and does not express the stem-loop cassette(s). The plasmid can be propagated in bacteria under kanamycin selection.

FIG. 1D shows a schematic of DNA expression plasmids EGFP/Neo-diipMG and HyTK-pMG, modified to express multiple copies of the U6 promoter and shRNA cassettes. Copies of the U6 promoter and shRNA cassettes could be introduced into this plasmid using the unique restriction enzymes, XhoI and Not I. The EGFP gene is under control of the human elongation factor (EF) 1 α hybrid promoter and NeoR or HyTK gene is under control of the CMV IE promoter. The bovine growth hormone (bGhpA) and late SV40 poly A sites (SV4OpA) are shown. The synthetic poly A and pause site (SpAn) and E. coli origin of replication are shown. The plasmid HyTK-pMG was generated from pMG (InvivoGen, San Diego, Calif.) using site-directed mutagenesis to remove a Pac I RE site at position 307 and replacing the Hy gene with the HyTk fusion gene, which combines the hygromycin phosphotransferase gene with the herpes thymidine kinase (Tk) suicide gene. (Cooper, L. J., et al., 2003; Lupton, S. D., et al., 1991) The Neo-diipMG DNA vector was modified from HyTK-pMG by replacing the hygromycin phosphotransferase (Hy) gene with the neomycin phosphotransferase (NeoR) gene and removing intron A and IRES element by Bst XI and Spe I digestion. The plasmid EGFP/Neo-diipMG contains the enhanced green fluorescent protein gene (EGFP) blunt-end ligated into the unique Nhe I site under control of the hybrid EF1a promoter.

FIG. 1E shows ten DNA expression plasmids that vary in number of U6 promoter and 705 stem-loop cassettes from 1 to 10.

FIG. 1F shows a DNA expression plasmid that contains 6 copies of the U6 promoter and stem-loop cassette having a scrambled sequence.

FIG. 1G shows a schematic of a HLA A3 molecule and relative binding sites of siRNA antisense strand and PCR primers (not to scale). Signal peptide (sp) α1, 2, and 3 regions and cytoplasmic region are shown (as determined from SWISSPROT:1A03_HUMAN).

Kinetic analysis of expression of multiple copies of the hairpin-loop (U6shRNA) cassettes results in augmented down-regulation of classical HLA class I protein expression.

FIG. 2A is a graph showing cell surface expression of HLA ABC on Jurkat T cells transiently transfected. Transient Jurkat transfectants were analyzed for 5 days to determine the RNAi kinetics represented by the percentage loss of binding of PE-conjugated anti-HLA ABC. The percent down-regulation of HLA ABC of Jurkat cells transfected with EGFP/Neo-diipMG plasmid expressing 6 copies of the scrambled U6shRNA control cassette at each time point was less than 4% (data not shown). Maximal siRNA-effect 4 days after transfection fits the 1^{st} order polynomial for y=8.41×−7.06 (R²=0.94).

Transfection of Jurkat cells with 5 mg of DNA plasmid, linearized at unique Pac I RE site, was achieved by electroporating 100 mL of Jurkat cells at 30×10⁶/mL in Nucleofector Solution V using a Nucleofector I electroporator device operating under program #T-14, per manufacturer's conditions. (Amaxa GmbH, Cologne, Germany) To achieve stable transfections, cytocidal concentration of G418 sulfate (Calbiochem, La Jolla, Calif.) at 1 mg/mL was added 72 hours after electroporation. Transfection of primary HLA A2⁺ human T cells in PBMC was achieved 3 days after stimulation with 30 ng/mL of OKT3 by electroporating with a single pulse of 250 V for 40 msec 400 mL of 20×10⁶/mL T cells in hypo-osmolar buffer using a Multiporator device (Eppendorf AG Hamburg, Germany) with 5 mg of linearized DNA plasmid. (Cooper, L. J., et al., 2003) Following electroporation, cytocidal concentrations of G418 (0.8 mg/mL) or hygromycin (0.2 mg/mL, InvivoGen) were added on day 5 of each 14-day culture cycle.

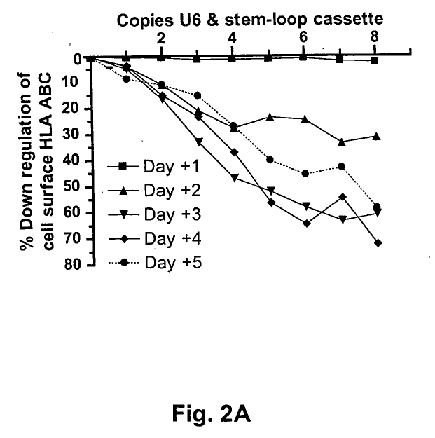

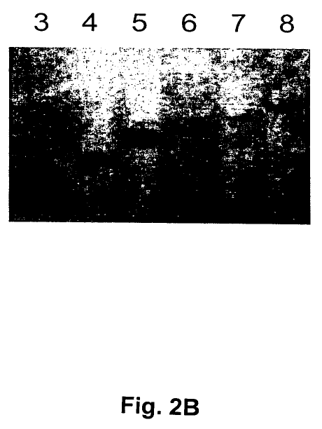

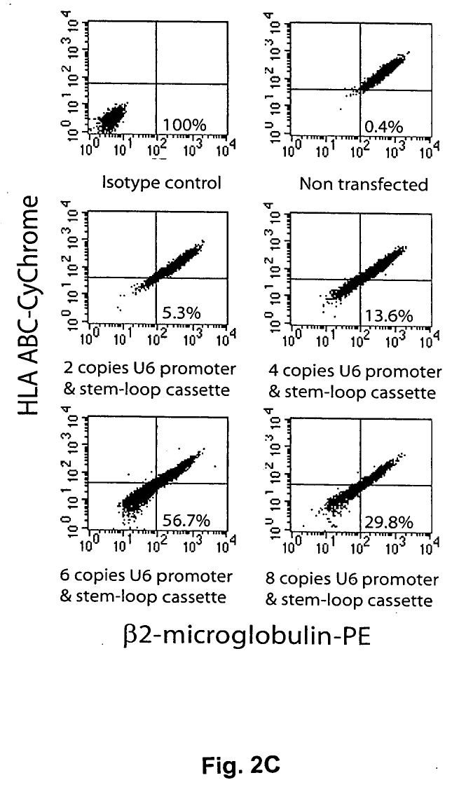

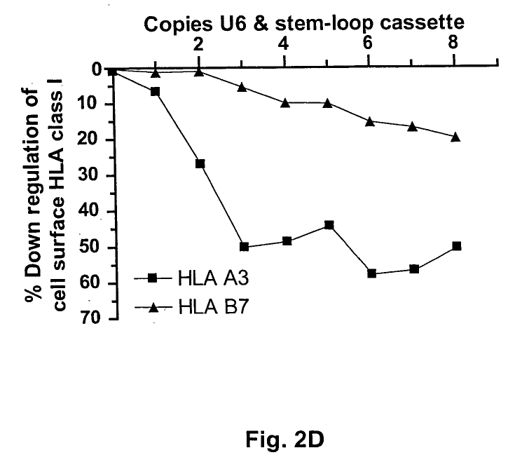

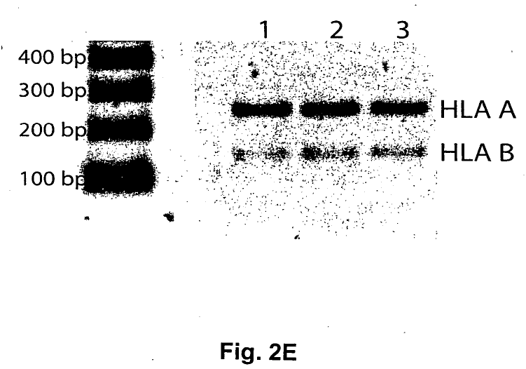

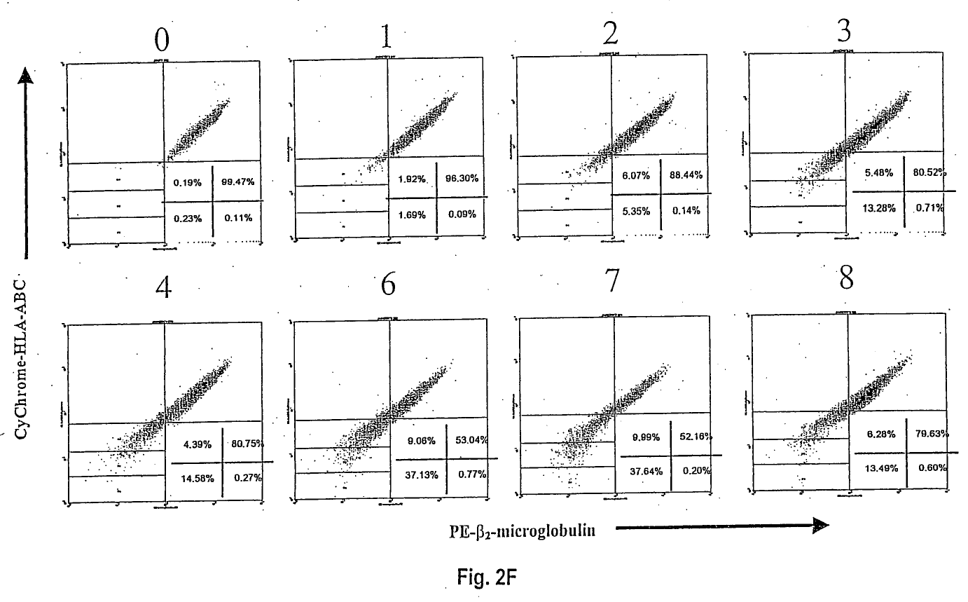

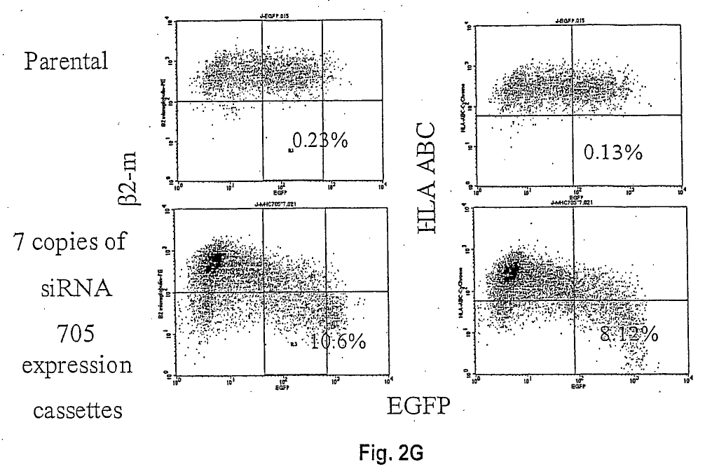

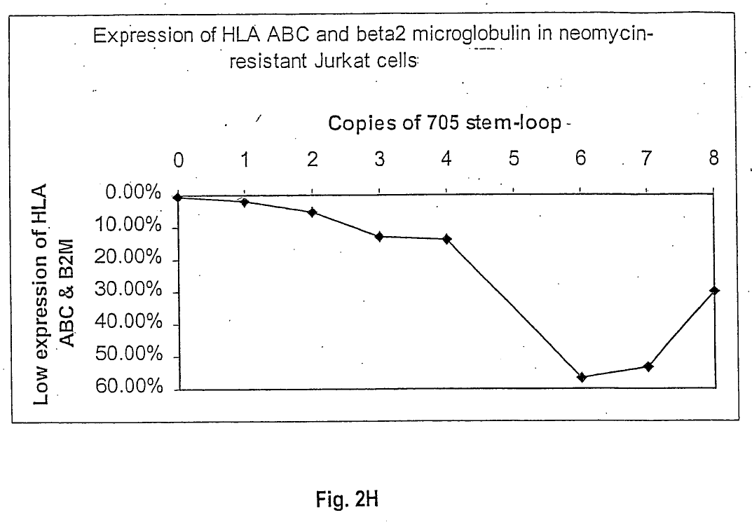

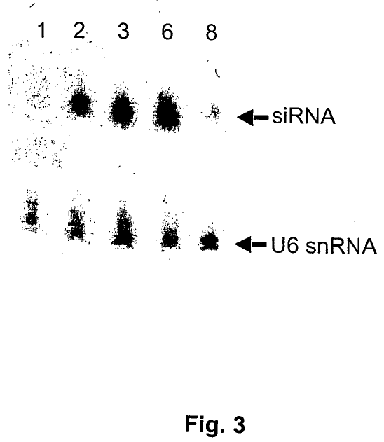

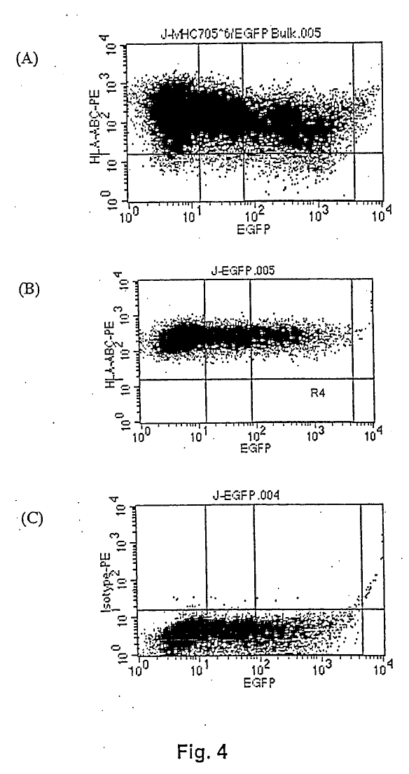

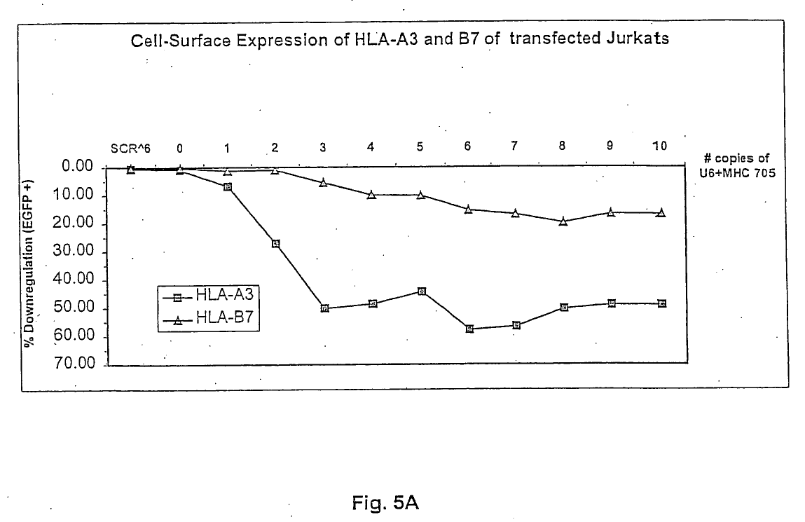

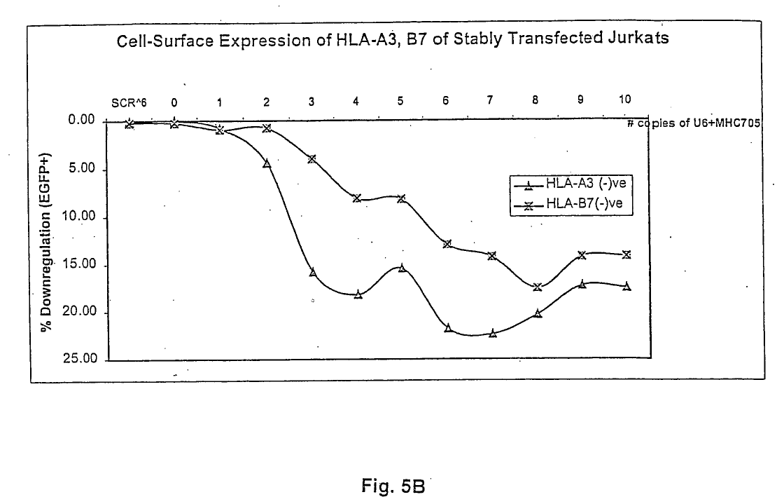

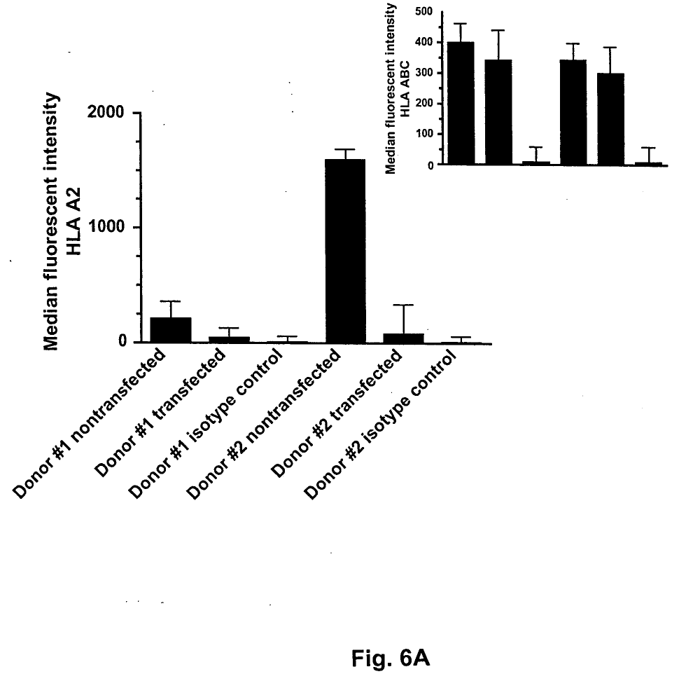

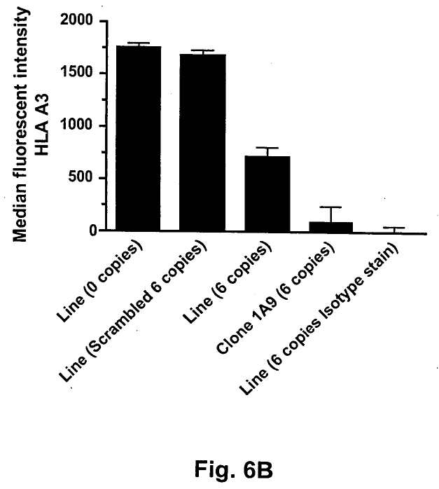

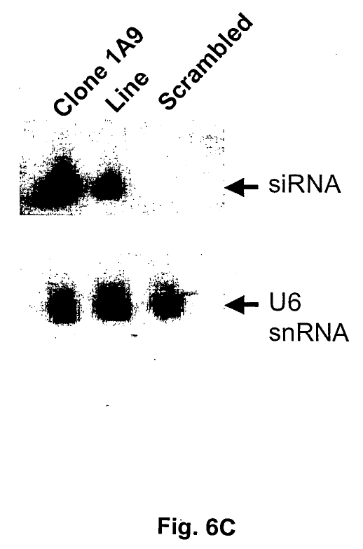

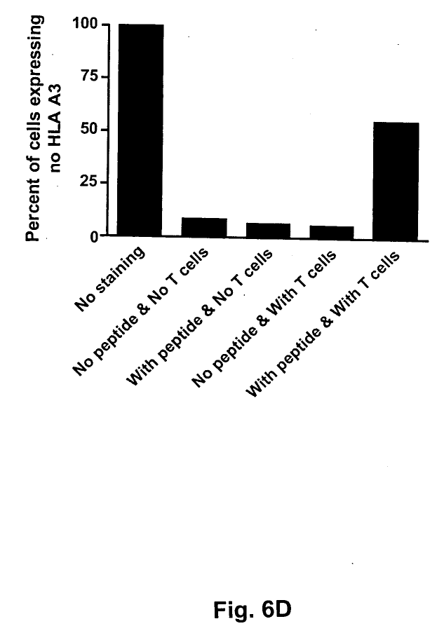

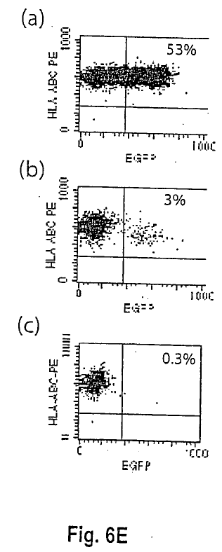

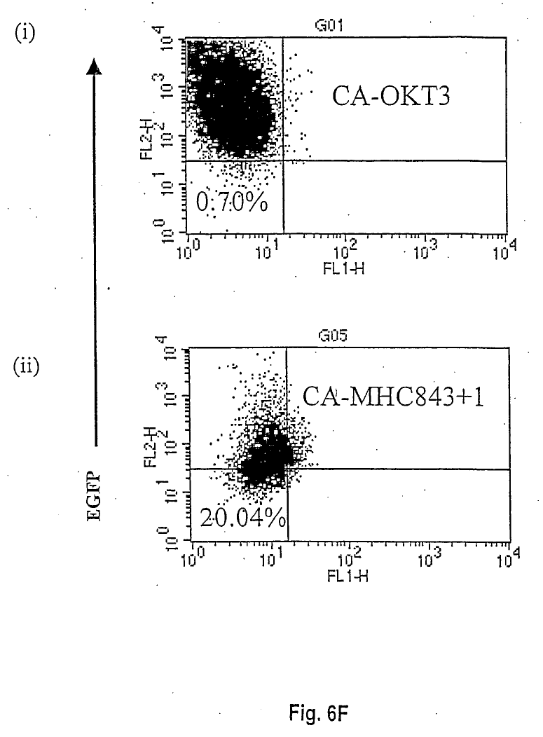

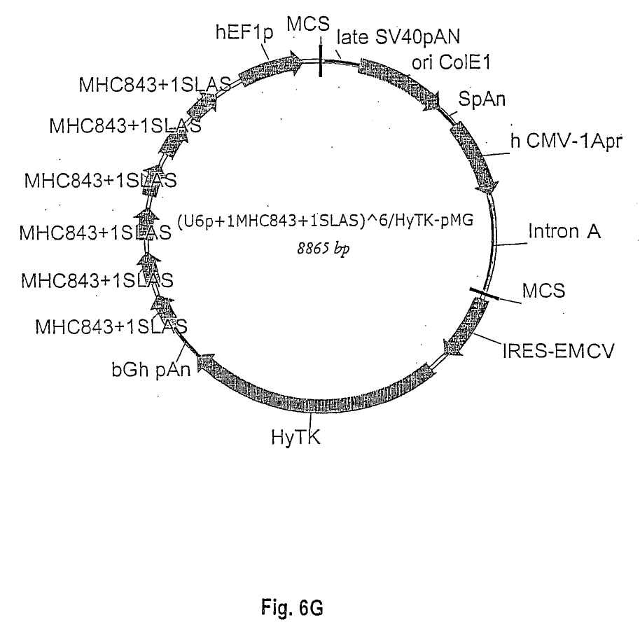

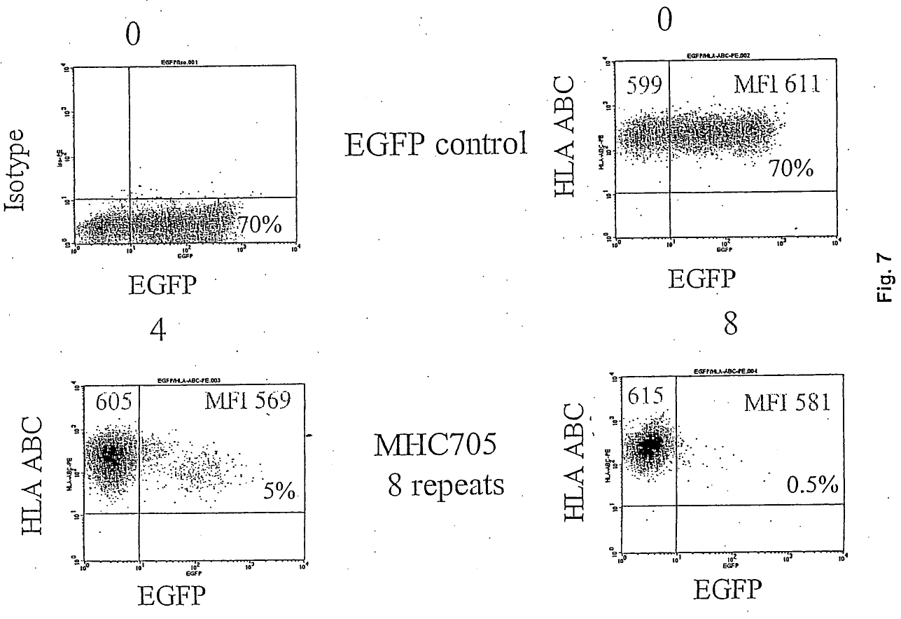

FIG. 2B is a gel photograph of the results of a Southern blot analysis demonstrating integration of plasmids bearing U6shRNA cassettes. G418-resistant genetically modified Jurkat cells were transfected with up to 8 copies of the anti-HLA ABC U6shRNA cassette. U6shRNA cassette copy number is indicated. The cells were probed with an approximately 320 bp fragment encompassing bovine growth hormone poly A plasmid sequence, released by Bam HI and Eco RI digest. U6 promoter and stem-loop shRNA cassette copy number is indicated.

Expression of multiple copies of the U6shRNA cassettes results in augmented and durable down regulation of classical HLA class I protein expression. In FIG. 2C, G-418-resistant Jurkat cells transfected with EGFP/Neo-diipMG plasmid with 0 to 8 copies of the U6shRNA cassette were analyzed by multiparameter flow cytometry for binding of PE-conjugated anti-b₂m (x-axis) and CyChrome-conjugated anti-HLA ABC (y-axis) non-covalently expressed with soluble β₂-microglobulin on the cell surface, on EGFP⁺ cells. The binding of isotype control mAbs is shown. The percentage of cells in the lower left quadrant (HLA ABC^{low}b₂m^{low}) is shown for each plot.

Augmented siRNA expression from multiple U6shRNA cassettes can help overcome adverse siRNA position effects. FIG. 2D is a graph showing the expression of HLA A3 and B7 on G418-resistant Jurkat cells transfected with EGFP/Neo-diipMG plasmid containing 0 to 8 copies of the HLA ABC-specific shRNA cassettes. Transfectants were analyzed by multiparameter flow cytometry for binding of biotin-conjugated anti-HLA A3 and anti-HLA B7 on EGFP⁺ cells.

FIG. 2E is a gel photograph showing the relative levels of HLA A and HLA B mRNA from Jurkat cells that were not transfected (lane 1), transfected with EGFP/Neo-diipMG plasmid (lane 2), or transfected with EGFP/Neo-diipMG plasmid modified to express 6 copies of the scrambled shRNA (lane 3), and amplified by RT-PCR using HLA A- and HLA B-specific primers and resolved by agarose gel electrophoresis. Densitometry revealed that the ratio of HLA A repliconto HLA B replicon was approximately 3:1 (data not shown).

FIGS. 2F, 2G and 2H show cell surface expression of HLA ABC on Jurkat T cells stably transfected with DNA expression plasmids which vary in number of U6 promoter and stem loop cassettes from 0 to 8.

FIG. 3 is a gel photograph showing the results of Northern blot analysis of siRNA. Expression levels of shRNA in G418-resistant genetically modified Jurkat cells transfected with up to 8 copies of the U6 promoter and HLA ABC-specific shRNA, probed using an oligonucleotide complementary to the antisense strand of the shRNA. An oligonucleotide complementary to the endogenous U6 small nuclear (sn) RNA was used as an internal RNA loading standard. The U6shRNA cassette copy number is indicated.

FIG. 4 shows the results of flow cytometry analyses of expression of EGFP and/or binding of PE-conjugated anti-HLA ABC to Jurkat T cells transfected with a DNA plasmid expressing 6 copies of the U6 promoter and 705 stem-loop cassette (FIG. 4A) or DNA plasmid EGFP/diipMGNeo (no U6 promoter or stem-loop cassette) (FIGS. 4B and C).

FIG. 5 shows the results of flow cytometry analyses of cell-surface expression of HLA A3 and HLA B7 on Jurkat T cells transiently transfected (FIG. 5A) or stably transfected (Jurkat cells resistant to cytocidal concentration of neomycin) (FIG. 5B) with a DNA plasmid expressing from 0 to 10 copies of the U6 promoter and 705 stem-loop cassette.

FIGS. 6A-G show phenotypic effects of HLA A-specific siRNA in Jurkat clone and differentiated primary human T cells. FIG. 6A shows the down-regulation of cell-surface HLA A2 (and HLA ABC, insert) protein expression on hygromycin-resistant heterozygous (donor #1, HLA*A 0201/0301, B*0702/1402) or homozygous (donor #2, HLA A*0201/0201, B*0702/3503) HLA-A2⁺ primary T cells transfected with a HyTK-pMG DNA plasmid modified to express 6 copies of the shRNA cassette. T cells were analyzed by flow cytometry for binding of PE-conjugated anti-HLA-A2 and HLA ABC. Dead cells were excluded by uptake of propidium iodide (PI). Jurkat and primary T cells were maintained in T-cell media: RPMI 1640 (Irvine Scientific, Santa Ana, Calif.) supplemented with 2 mM L-Glutamine (Irvine Scientific, Santa Ana, Calif.), 25 mM HEPES (Irvine Scientific), 100 U/mL penicillin, 0.1 mg/mL streptomycin (Irvine Scientific) and 10% heat-inactivated defined fetal calf serum (FCS) (Hyclone, Logan, Utah). Some Jurkat transfectants were cloned by limiting dilution in 96-well plates after sorting for loss of class I HLA expression. Primary T cells were expanded from peripheral blood mononuclear cells (PBMC) derived from healthy volunteers using previously described methods. (Cooper, L. J., et al., 2003) Typically, 1×10⁶ T-cells were restimulated every 14 days by adding 30 ng/mL anti-CD3 (OKT3, Ortho Biotech, Raritan, N.J.), 10⁷ irradiated PBMC and 10⁷ irradiated LCL. Recombinant human IL-2 (Chiron, Emeryville, Calif.) at 25 U/mL was added every 48 hours, beginning on day 1 of culture.

FIG. 6B is a graph showing the identification of clone expressing siRNA with low levels of HLA A3. Binding of HLA A3-specific mAb and isotype control mAb to Jurkat T-cell clone, 1A9, transfected with the EGFP/Neo-diipMG plasmid modified to express six copies of the HLA ABC-specific shRNA cassette, and the line from which it was derived. Other lanes show transfected Jurkat cell lines expressing no shRNA or the scrambled version. The level of HLA A3 cell-surface expression was measured by flow cytometry on EGFP⁺PI^{neg} cells using biotin-conjugated mAb specific for HLA A3 and PE-conjugated streptavidin and is described by the MFI±CV. The binding of an isotype control mAb demonstrates the minimal MFI.

The PE-conjugated and CyChrome-conjugated (clone G46-2.6) mAbs specific for HLA ABC (BD Biosciences, San Jose, Calif.) were used at a dilution of 1:20. The biotin-conjugated mAbs specific for HLA A3 and HLA B7/B27 (One Lambda Corporation, Canoga Park, Calif.) were used at a dilution of 1:20. Staining of cells was accomplished in Hank's Balanced Salt Solution (HBSS) (Irvine Scientific) containing 2% FCS. In some experiments, non-viable cells that had taken up 1 μg/mL propidium iodide (PI) were excluded from analysis. Data acquisition was performed on a FACScan (BD Biosciences) and the percentage of cells in a region of analysis, median fluorescent intensity (MFI), and coefficient of variation (CV) were calculated using CellQuest version 3.3 (BD Biosciences). Fluorescent activated cell sorting, MoFlo MLS (Dako-Cytomation, Fort Collins, Colo.), was performed on Jurkat cells genetically modified with the EGFP/Neo-diipMG plasmid expressing 6 copies of the U6- HLA ABC-specific shRNA cassette.

FIG. 6C shows the results of a Northern blot analysis of siRNA. Jurkat clone 1A9, the line expressing 6 copies of the shRNA cassette from which the clone was derived, and G418-resistant Jurkat cells transfected with 6 copies of the scrambled shRNA cassette.

FIG. 6D shows that HLA A3^{neg} cells transfected with HLA ABC-specific siRNA are protected from T cell-mediated specific lysis. The percentage of P^{neg} EGFP⁺ HLA A3^{neg} target cells relative to PI^{neg} EGFP⁺ HLA A3⁺ cells were measured after incubating the HLA A3-restricted peptide RLRPGGKKK [SEQ ID NO: 4]-pulsed 1A9 Jurkat clone with HIV-specific HLA A3-restricted T-cell clone 28A2-15.

FIG. 6E shows (a) expression of EGFP and HLA ABC in G418-resistant T cells that are genetically modified with EGFP/Neo-diipMG DNA that does not express siRNA. Selective loss of EGFP⁺ expression in G418-resistant primary human T cells transfected with EGFP/Neo-diipMG DNA plasmid expressing (b) 4 or (c) 6 copies of the U6shRNA specific for HLA ABC. Percentage of HLA ABC⁺ EGFP⁺ cells in the top-right quadrant is displayed.

FIG. 6F shows the percent down-regulation of HLA class I in CA-OKT3 T cells and CA-MHC843+1 T cells.

FIG. 6G shows a DNA expression plasmid containing 6 copies of the U6 promoter and “843+1” stem-loop cassette. The stem-loop cassette DNA sequence is 5′CACCTGCCATGTGCAGCATGAtttgtgtagTCATGCTGCACATGGCAGGTG3′ [SEQ ID NO: 3].

FIG. 7 shows the results of flow cytometry analyses of EGFP expression in neomycin-resistant primary T cells stably transfected with 8 copies of the U6 promoter and 705 stem-loop cassette.

### DETAILED DESCRIPTION OF THE INVENTION

The present invention relates in one aspect to a method for amplifying RNAi expression, preferably siRNA or shRNA expression, and the si/shRNA effect. This can be achieved, in a preferred embodiment, by increasing the number of promoters, preferably Pol III promoters, and double-stranded RNA cassettes in a DNA expression vehicle such as a plasmid. As shown in the Examples, an application of this approach is demonstrated by down-regulating HLA gene expression in human T cells.

It has been previously demonstrated that introducing viral immune evasion genes can modulate immune recognition by blocking expression of classical HLA class I molecules (York, I. A., et al., 1994; Lorenzo, M. E., et al., 2001; Berger, C., et al., 2000; Barel, M. T., et al., 2003). However, since siRNAs are a small nucleic acid reagent that, in contrast to virally-derived proteins, are unlikely to elicit an immune response, one embodiment of the present invention is directed to expressing intracellular siRNAs, homologous to a sequence conserved in most classical polymorphic HLA-A, -B and -C loci, as hairpin transcripts from mammalian RNA polymerase III (Pol 111) promoters (Lee, N. S., et al., 2002; Brummelkamp, T. R., et al., 2002) to achieve suppression of major histocompatibility complex (MHC) class I cell-surface expression.

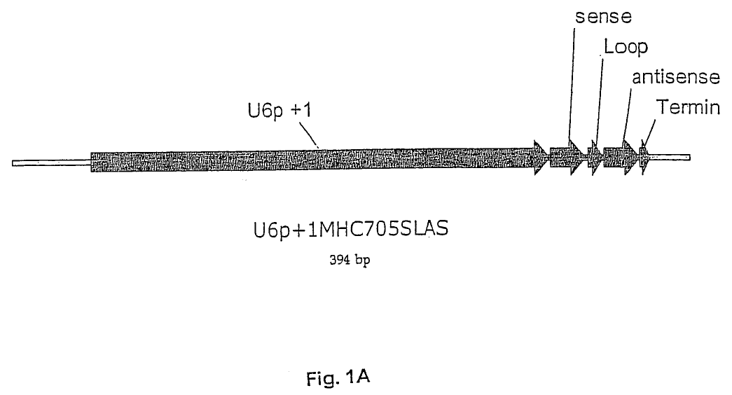

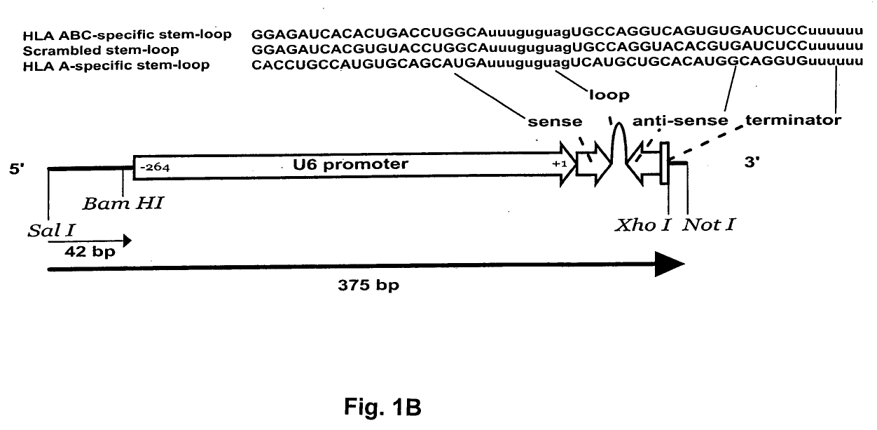

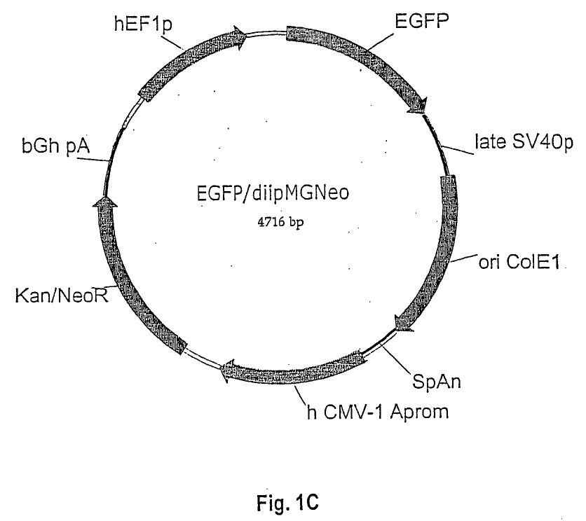

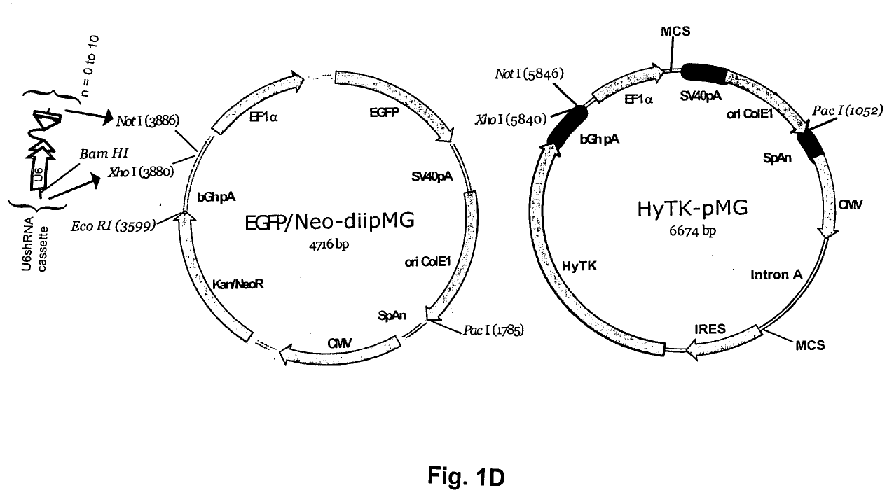

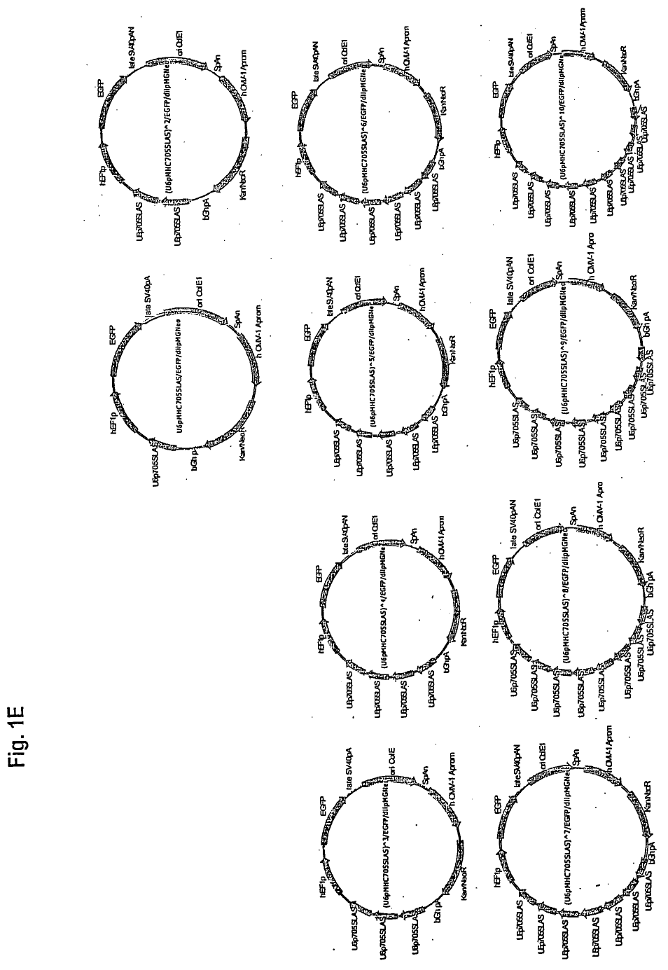

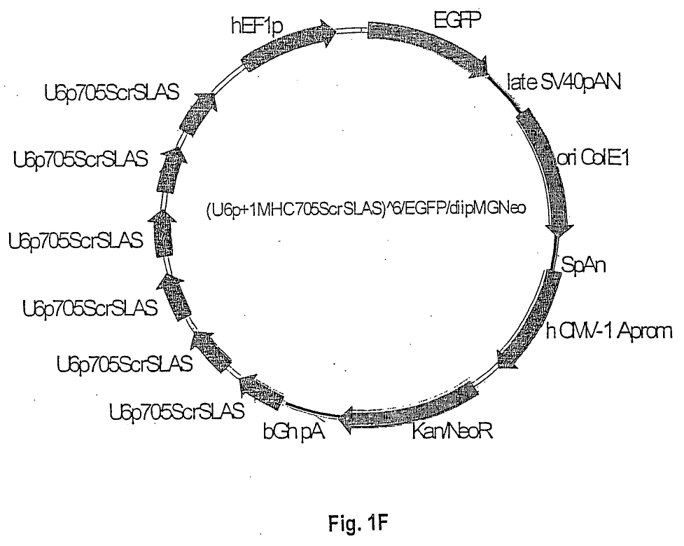

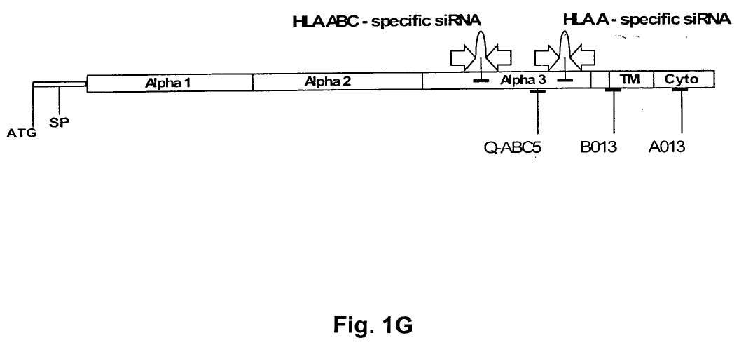

Since the design of HLA ABC-specific siRNA is constrained by choosing 21 -basepair (bp) binding-sites homologous to the majority of classical class I alleles, which may include sites associated with adverse siRNA position effects, and since multiple endogenous genes need to be simultaneously targeted to achieve down regulation of HLA molecules, a system was developed to titrate/augment expression of shRNA preferably using a plasmid vector by preferably titrating the number of U6 promoters and shRNA cassettes (FIG. 1B and 1D). FIG. 1G depicts an HLA class I molecule and the relative position of the siRNA binding.

To titrate/augment RNAi-effects, MHC class I expression on Jurkat cells, a T-cell line expressing HLA A*0301/0301 B*0702/3503 Cw*401/0702, transfected with a panel of DNA vectors containing between 0 and 8 copies of the U6shRNA cassette was transiently down regulated. A flow cytometry kinetic study demonstrated that the down-regulation of HLA ABC antigens peaked between three to four days after transfection (FIG. 2A), reflecting the time required to achieve sufficient shRNA expression and RNAi to prevent replacement of HLA A, HLA B and HLA C molecules on the cell-surface. Increasing the copy-number of the U6shRNA cassettes from 1 to 8 resulted in a steady increase in RNAi, with a maximal 19-fold improvement in the siRNA-effect. Down-regulation of HLA ABC expression was specific as cells transfected with a DNA plasmid expressing a scrambled version of the HLA ABC-specific shRNA showed negligible loss of HLA class I cell-surface expression (FIG. 6B and data not shown).

Durable down regulation of HLA ABC levels was achieved as a result of augmented shRNA expression. While expression of two copies of the U6shRNA cassettes resulted in 5.3% of the G418-resistant T cells with down-regulated protein expression of both HLA ABC and β₂-microglobulin (β₂-m), this percentage increased approximately 11-fold when 6 copies of the U6shRNA cassettes were expressed (FIG. 2C). Southern blotting analyses confirmed that the G418-resistant Jurkat cells had integrated the correct number of U6shRNA cassettes (FIG. 2B). The siRNA-mediated down-regulation of HLA ABC has been maintained for an extended period of time, as transfected Jurkat cells continue to demonstrate down-regulation of HLA ABC protein expression after 6 months of passage in tissue culture (data not shown). No β-IFN production, a potentially deliterious and non-specific effect induced by expression of shRNA, was detectable in the cells expressing multiple copies of the U6shRNA cassettes (data not shown).

The degree of HLA ABC protein down-regulation correlated with the level of expression of stem-loop dsRNA as confirmed by Northern analyses of the shRNA constructs (FIG. 3). The ability to down-regulate HLA ABC protein expression peaked with the introduction of 6 copies of the shRNA cassettes in stable transfectants (FIG. 2C), while 7 to 10 copies of the shRNA cassettes showed a slight decrease in HLA down-regulation, which was consistent with a relative decline in their intracellular RNA expression (FIG. 3 and data not shown). This reason(s) for this loss in efficacy and expression with greater than 6 copies of the cassette are not clear, but could include local chromatin alterations resulting in relative loss of Pol m expression or a selective disadvantage of stable over-expression of anti-HLA ABC shRNA. The peak siRNA-effect may be different for other targets. The ability to titrate the U6 promoter and shRNA cassettes allows investigators to titrate the copies of the introduced cassettes with the degree of gene down-regulation.

There may be differential inhibitory effects for individual MHC class I alleles, indicative of position-dependent siRNA-effects, despite the similarity of the HLA nucleic acid sequences, leading to selective down-regulation of individual alleles, which could not be detected using a pan-HLA class I-specific monoclonal antibody (mAb). Therefore, the cell-surface expression of HLA A3 and HLA B7 was determined using allele-specific mAbs. Increasing the number of HLA ABC-specific U6shRNA cassettes resulted in a much greater degree of down-regulation of cell-surface HLA A3 protein-expression relative to HLA B7 protein expression (FIG. 2D). This was surprising since Jurkat cells are homozygous for HLA A3 and appear to express 3-fold more HLA A mRNA than HLA B (FIG. 2E) (Hakem, R., et al., 1989). Given the variability observed in target propensity to RNAi-effects, it is likely that the individual susceptibilities of HLA A and B gene expression to inhibition may differ due to the different sequence contexts flanking the RNAi target site, which are currently not predictable from the nucleotide sequence. Despite this possibility of adverse nucleotide position effects, increasing the number of U6 promoter and HLA ABC-specific stem-loop cassettes from 1 to 6 copies still improved the down regulation of HLA B7 by 13-fold, indicating that increasing shRNA expression can be used as a strategy to help overcome the consequences of target position dependency of RNAi.

To test if transfected cells that have down regulated classical HLA class I molecules due to a siRNA-effect are capable of avoiding immune recognition by HLA-restricted antigen-specific αβTCR⁺CD8⁺cytotoxic T lymphocytes (CTL), a Jurkat clone (1A9) with high expression levels of the shRNA and a consequent low-level of HLA A3 expression (FIG. 6B and 6C) was isolated. Flow cytometry demonstrated that this clone had 97% reduced HLA A3 cell-surface protein expression, compared with cells expressing no shRNA. To test for protection against a CTL-response, the Jurkat clone was exogenously loaded with a saturating concentration of the HLA A3-restricted peptide RLRPGGKKK [SEQ ID NO: 4] (derived from the p17 sub region of HIV gag), which is recognized by the HLA A3⁺cytolytic T-cell clone, 28A2-15 (Lewinsohn, D. A., et al., 2002). The peptide-loaded 1A9 cells were co-cultured with 28A2-15, and after 4 hours the expression of HLA A3 on surviving Jurkat cells was analyzed by flow cytometry. After the co-culture of T cells with a peptide-loaded Jurkat clone, the percentage of EGFP⁺Jurkat cells that were devoid of HLA A3 cell-surface expression increased 7-fold from 8.5% to 56% (FIG. 6D), as this assay selects for cells that have essentially lost expression of HLA A3, given that CTL can be activated for cytolysis by as few as 1 to 100 cell-surface MHC molecules (Sykelev, Y., et al., 1996).

To demonstrate the activity of shRNA in primary T cells and avoid auto-deletion of T cells that had lost expression of classical HLA class I molecules by autologous NK-T cells present in PBMC, a new shRNA was constructed with a 21 nucleotide sequence completely homologous to most HLA A alleles, but which contained bp mismatches with HLA B and C alleles. To generate HLA A2^{neg} T cells that could be eliminated in vivo by ganciclovir-mediated ablation, heterozygous and homozygous HLA A2⁺primary T cells were transfected with the HyTK-pMG plasmid, modified to express 6 copies of the HLA A-specific shRNA (FIG. 1D). Hygromycin-resistant T cells could be demonstrated to have down-regulated HLA A2-expression, relative to drug-resistant parental T-cell controls that do not express the shRNA (FIG. 6A). As expected, there was only a small decrease in the binding of the mAb specific for HLA ABC to the T cells that had down-regulated HLA A2 expression, reflecting the fact that this mAb clone recognized an epitope also present on HLA B and C molecules (FIG. 6A insert). This is believed to be the first demonstration of siRNA-effects in primary T cells electroporated with a DNA plasmid.

The ability to disrupt antigen presentation by down-regulating HLA gene expression using RNAi is an approach to avoiding T cell-mediated immune recognition, which might be used to facilitate transplantation and/or adoptive immunotherapy between HLA-divergent individuals or to prolong the in vivo survival of transferred T cells that express vector-ecoded immunogenic transgenes.

The embodiment of the present invention for introducing successive U6shRNA cassettes into an expression-vector should be generally useful for other applications in which controllable levels of RNAi-mediated target knockdown are desired and this technology should help address limitations to RNAi-induced gene silencing that depend on achieving adequate intracellular levels of siRNA (Cooper, L. J., et al., 2003). Additional changes can be envisioned to further improve the efficacy of the siRNA vector, such as using enhancer elements, alternative Pol III promoters, alternative promoters, and a combination of siRNAs directed to different regions of the HLA genes and/or targeting multiple essential components of antigen processing and MHC (class I and II) expression.

In one embodiment, increasing the number of promoter and RNAi (si/shRNA) encoding cassettes, preferably U6 promoter and stem-loop expression cassettes, in an expression vector can significantly increase the amount of RNAi (si/shRNA) leading to significantly improved down-regulation of a target gene, such as those encoding HLA molecules.

In the present invention, increasing the number of U6 promoter and si/shRNA stem-loop expression cassettes in an expression vector resulted in increased si/shRNA expression. Thus, in a preferred embodiment, by varying the number of promoters, preferably a U6 promoter, and stem-loop cassettes in an expression vector, desired levels of down-regulation of a target gene can be achieved.

A RNAi- (or si/shRNA-) expressing concatamer means generally an expression vehicle containing multiple units capable of expressing RNAi (si/shRNA). The preferred concatamer is in the form of an expression vector, as illustrated in the figures.

A promoter-RNAi expression cassette is a cassette containing a promoter sequence, preferably operatively linked to one or more DNA sequences encoding a sense strand and/or antisense strand of a RNAi (si/shRNA) molecule. The cassette preferably contains DNA sequences encoding the sense and antisense strands in the form of a single hairpin or stem-loop sequence. Examples are shown in Example 3 and FIGS. 1A and 1B.

The concatamer may comprise any multiple number of expression units or cassettes capable of enhancing RNAi (si/shRNA) expression. A plurality of expression cassettes preferably includes 2-10 cassettes, but also may include more than 10, such as 11-15 or 16-20. The number of expression cassettes are preferably 2-5 or 6-8, and more preferably 2, 3, 4, 5, 6, 7 or 8, and most preferably 6. The number of expression cassettes can be decreased or increased depending on the degree of titration desired.

The promoter can be any suitable promoter, including a Pol III promoter such as U6 or VA1 promoter.

In a preferred embodiment, an expression vector can be fashioned to express si/shRNA, an antibiotic resistance gene to select stable transfectants, and a reporter gene.

In another embodiment, conservative changes to the DNA sequence of introduced HLA genes, preferably HLA class I genes, which do not alter the amino acid sequence but prevent pairing with the introduced si/shRNA, may allow one to alter HLA expression in cells expressing si/shRNA specific for endogenous HLA (preferably class I) genes.

The present invention has been shown to down-regulate HLA gene expression, which can therefore avoid immune recognition to facilitate transplantation and/or adoptive immunotherapy between HLA-divergent individuals.

In the present invention, si/shRNA sequences thus can be identified that down-regulate gene expression, including sequences that specifically down-regulate HLA class I gene expression. In accordance with one embodiment of the present invention, double-stranded RNA are chemically synthesized (Dharmicon) and used to transfect U293T cells (primary human embryonal kidney line) to down-regulate classical HLA class I gene expression or selected HLA gene expression. Cells can be transfected with about 100 nM to about 200 nM of the selected double-stranded RNA, preferably formulated in oligofectamine (Invitrogen). In another embodiment, the double-stranded RNA can be produced in the cell, preferably with an expression vector.

In the present invention, DNA plasmids can be modified to contain a user-defined number of U6 and stem-loop cassettes (FIGS. 1A-F). This was shown when cells transfected with a panel of DNA vectors expressing increasing numbers of U6 promoter and stem-loop cassettes resulted in increased down-regulation of the target gene, including in one embodiment a HLA class I gene. This was demonstrated with transiently transfected Jurkat cells (FIG. 5A) as well as stable transfectants (FIG. 5B). The transient assay demonstrates that the time (4 days) to achieve maximal down-regulation of HLA class I gene expression is consistent with the time needed to interrupt gene expression by expression of si/shRNA and for the MHC molecules to be lost from the cell-surface. Specificity for the si/shRNA sequence is indicated by the fact that cells transfected with a DNA plasmid expressing a scrambled version of the stem-loop do not show loss of HLA class I expression.

Cells transfected with a panel of DNA vectors expressing increasing numbers of U6 promoter and stem-loop cassettes were demonstrated to have increasing expression of si/shRNA by Northern blotting (FIG. 3).

Cells transfected with a DNA vector expressing the stem-loop cassette maintain the down regulation of HLA class I molecules for greater than 6 months (FIG. 4A).

Cells exhibiting down-regulated expression of HLA class I molecules can be coned, thus demonstrating that the expression of multiple copies of the U6 promoter and stem-loop cassettes is a stable phenotype.

Increasing the expression of si/shRNA through expressing multiple copies of the U6 promoter and stem-loop cassettes can help overcome the loss of si/shRNA efficacy associated with selection of a target sequence that is constrained by positional effects. This was demonstrated by selectively analyzing the cell-surface expression of HLA-A3 and HLA-B7, two HLA class I alleles on Jurkat cells using a monoclonal antibody (mAb) specific for HLA-A3 and HLA-B7 (FIG. 5).

Primary human T cells were transfected with the DNA plasmid expressing multiple copies of the U6 promoter and stem-loop cassettes and loss of an HLA class I gene were demonstrated (FIGS. 6F-G). In this experiment a different stem-loop cassette, designated 843+1, was used to target just the HLA-A locus, instead of all HLA class I genes. This was necessary since previous experiments transfecting primary T cells with DNA plasmids containing multiple copies of the U6 promoter and 705 stem-loop cassette in the DNA plasmid EGFP/diipMGNeo resulted in the generation of neomycin-resistant HLA class I-positive T cells that expressed very low levels of EGFP (FIG. 7). This is consistent with the auto-deletion of transfected T cells that down-regulated HLA class I molecules and were deleted by natural killer (NK)-T cells.

The present invention is useful for both in vitro and in vivo applications, including in humans.

The term “introducing” encompasses a variety of methods of introducing DNA into a cell, either in vitro or in vivo, such methods including transformation, transduction, transfection, and infection. Vectors are useful and preferred agents for introducing DNA encoding the interfering RNA molecules into cells. Possible vectors include plasmid vectors and viral vectors. Viral vectors include retroviral vectors, lentiviral vectors, or other vectors such as adenoviral vectors or adeno-associated vectors.

Alternative delivery systems for introducing DNA into cells may also be used in the present invention, including, for example, liposomes, as well as other delivery systems known in the art.

Methods for introducing the concatamers and expression vectors of the invention into cells such as mammalian cells are otherwise readily known in the art, some of which are described in co-pending U.S. application Ser. No. 10/365,643, filed Feb. 13, 2003, and Ser. No. 10/629,895, filed Jul. 30, 2003, which are fully incorporated herein by reference.

The present invention is further illustrated by the following examples which are not intended to be limiting.

### EXAMPLE 1

MHC class I gene expression on U293T cells transfected with synthetic double-stranded RNA specific for conserved HLA class I sequence was examined for down-regulation. U293T cells were plated in log-phase growth and transfected with 218 nM of double-stranded RNA suspended in oligofectamine (Invitrogen). After 96 hours, the cell-surface expression of HLA class I was determined by flow cytometry using FITC-conjugated anti-HLA ABC (PharMingen).

HLA A2 gene expression on U293T cells transfected with synthetic double-stranded RNA specific for conserved HLA class I sequence also was examined for down-regulation. U293T cells were plated in log-phase growth and transfected with 218 nM of double-stranded RNA suspended in oligofectamine (Invitrogen). After 96 hours, the cell-surface expression of HLA class I was determined by flow cytometry using FITC-conjugated anti-HLA A2 (PharMingen).

Examples of siRNA molecules that can reduce the cell surface expression of MHC class I molecules are:

[SEQ ID NO: 10]

MHC_ClassI_711: CA-CACUGACCUGGCAGCGGGAdTdG

[SEQ ID NO: 11]

MHC_ClassI_592: AA-CGGGAAGGAGACGCUGCAGdTdT

[SEQ ID NO: 12]

MHC_ClassI_238: CA-CAGACUCACCGAGUGGACCdTdG

[SEQ ID NO: 13]

MHC_ClassI_844: CA-CCUGCCAUGUGCAGCAUGAdTdT

The candidate nucleotide sequences containing shared homology were identified based upon comparisons of the published cDNA sequences. Flow cytometry showed that MHC_ClassI_{—}711 was able to reduce expression of HLA A, B, and C molecules, while MHC_ClassI_{—}238 and MHC_ClassI_{—}844 were able to reduce expression of HLA A2. MHC_ClassI_{—}592 had minimal effect on HLA A, B and C expression (and a small decrease in HLA A2 expression).

### EXAMPLE 2

U6 pol III promoter was joined to the 705 stem-loop sequence (GGAGATCACACTGACCTGGCAtttgtgtagTGCCAGGTCAGTGTGATCTCC [SEQ ID NO: 1]) or scrambled 705 stem-loop sequence (GGAGATCACGTGTACCTGGCAtttgtgtagTGCCAGGTACACGTGATCTCC [SEQ ID NO: 2]) by PCR. The 705 stem-loop sequence is complimentary to almost all human class I genes. This sequence is conserved in almost all HLA classical (and some non-classical) class I genes. The correct sequence was verified (data not shown).

### EXAMPLE 3

As shown in FIG. 1F, a DNA expression plasmid was constructed to contain and express 6 copies of the U6 promoter and stem-loop cassette having a scrambled sequence. The U6 PCR cassette was constructed to have Sal 1 and Xho 1 compatible restriction sites at its 5′ and 3′ ends, respectively. The cassette was cloned into the unique Xho I site of the EGFP/diipMGNeo expression vector, destroying the 5′ Sal 1 site with a Sal 1/Xho 1 ligation and recreating a unique Xho 1 site at the 3′ end. This new Xho 1 site was used for subsequent clonings of additional U6 cassettes using the same cloning strategy. In addition to the multiple U6 shRNA cassettes, the expression vector contains an EGFP reporter gene under control of the hybrid human elongation factor I (ELF1) and a-region promoter in the pMG vector purchased from Invitrogen. The correct construction of the DNA plasmids was validated by RE digestion resolved on agarose gel and by DNA sequence analyses (data not shown).

### EXAMPLE 4

Cell surface expression of HLA class I gene on Jurkat T cells was examined after the cells were transiently (FIG. 2A) and stably (FIGS. 2F, G and H) transfected with DNA expression plasmids in which the number of U6 promoter and stem-loop cassettes varied from 0 to 10. Transfection was achieved by non-viral gene transfer by electroporating 100 mL of Jurkat cells at 30×10⁶/mL Nucleofector™ Solution V (Amaxa) in a Nucleofector™ I electroporator device (Amaxa) using program # T-14 to achieve stably transfected cells, cytocidal concentrations of neomycin 72 hours after electroporation.

Jurkat cells transfected with DNA plasmid EGFP/diipMGNeo expressing between 0 to 8 copies of the U6 promoter and 705 stem-loop cassette (as well as DNA plasmid EGFP/diipMGNeo expressing 6 copies of the U6 promoter and scrambled 705 stem-loop cassette) were serially analyzed for up to 5 days post-electroporation by multiparameter flow cytometry and electronically gated for expression of EGFP. (FIG. 2A). Percentage loss of binding of PE-conjugated anti-HLA ABC on the transfected cells also was measured relative to the binding of PE-conjugated anti-HLA ABC on the untransfected (parental) Jurkat cells. Dead cells were excluded from analysis by uptake of 1 mg/mL propidium iodide (PI^{-ve}).

Neomycin-resistant Jurkat cells that were transfected with DNA plasmid EGFP/diipMGNeo expressing between 0 to 8 copies of the U6 promoter and 705 stem-loop cassette were analyzed by multiparameter flow cytometry and electronically gated on expression of EGFP. (FIG. 2F) Binding of PE-conjugated anti-b₂m (PharMingen) (x-axis) and CyChrome-conjugated anti-HLA ABC (PharMingen) (y-axis) also was measured relative to the binding of PE-conjugated anti-b₂m (PharMingen) and CyChrome-conjugated anti-HLA ABC on untransfected (parental) Jurkat cells. The percentage of cells in each quadrant (upper left=HLA⁺b₂m⁻, upper right=HLA⁺b₂m⁺, lower left=HLA⁻b₂m⁻, lower right=HLA⁻b₂m⁺) is shown for each plot.

Neomycin-resistant Jurkat cells that were transfected with DNA plasmid EGFP/diipMGNeo expressing 7 copies of the U6 promoter and 705 stem-loop cassette were analyzed by multiparameter flow cytometry and electronically gated on expression of EGFP. (FIG. 2G) Binding of PE-conjugated anti-b₂m (PharMingen) (x-axis) and CyChrome-conjugated anti-HLA ABC (PharMingen) (y-axis) also was measured relative to the binding of PE-conjugated anti-b₂m (PharMingen) and CyChrome-conjugated anti-HLA ABC on untransfected (parental) Jurkat cells. The percentage of cells that express EGFP and have down-regulated HLA class I gene expression is shown.

Neomycin-resistant Jurkat cells that were transfected with DNA plasmid EGFP/diipMGNeo expressing between 0 to 6 copies of the U6 promoter and 705 stem-loop cassette were analyzed by multiparameter flow cytometry and electronically gated on expression of EGFP. Percentage loss of binding of PE-conjugated anti-b₂m (PharMingen) and CyChrome-conjugated anti-HLA ABC (PharMingen) also was measured relative to the binding of PE-conjugated anti-b₂m (PharMingen) and CyChrome-conjugated anti-HLA ABC on untransfected (parental) Jurkat cells. Jurkat cells were maintained in RPMI 1640 (BioWhittaker, Walkersville, Md.) supplemented with 2 mM L-Glutamine (Irvine Scientific, Santa Ana, Calif.), 25 mM HEPES (Irvine Scientific), 100 U/mL penicillin, 0.1 mg/mL streptomycin (Irvine Scientific) and 10% heat-inactivated defined fetal calf serum (FCS) (Hyclone, Logan, Utah).

### EXAMPLE 5

A Northern blot analysis was conducted of RNA extracted from G418-resistant Jurkat cells stably transfected with DNA plasmids expressing up to 8 copies of the U6shRNA cassettes (FIG. 3). The constructs analyzed in lanes 1-5 contain 0, 2, 4, 6, and 8 shRNA cassettes, respectively. The RNA was isolated using RNA STAT-60 (TEL-TEST “B” Inc., Friendswood, Tex.) according to the manufacturer's instructions. 15 μg of total RNA was fractionated in 8M-6% PAGE, and electro-blotted for 2 hours onto Hybond-N+ membrane (Amersham Pharmacia Biotech). A ³²P-radiolabeled 21 bp probe complementary to the siRNA antisense strand was used for the hybridization reactions, which were performed for 16 h at 37° C. For size analysis, a 21-mer DNA oligonucleotide was electrophoresed alongside the RNA samples and used as a size control (not shown). The highest shRNA expression was detected with 6 copies of the shRNA cassette (lane 4). Other numbers of cassettes, including both higher and lower numbers, may achieve stronger expression in other experimental systems. Therefore, the optimal number of expression cassettes may and should be determined empirically for each selected targeted gene. A ³²P-radiolabeled 20-mer probe complementary to sequences of the endogenous U6 snRNA was used as a control for the amount and integrity of the RNA analyzed in each lane.

### EXAMPLE 6

During Southern blotting, 10 μg of genomic DNA were digested overnight with Eco RI and Not I RE, flanking the U6shRNA cassette(s). The DNA was run on a 0.8% agarose gel and denatured by soaking for 50 min at room temperature in 1.5 M NaCl, 0.5N NaOH. The gel was rinsed with water and neutralized at room temperature in 200-300 ml of 1 M Tris pH 8, 1.5 M NaCl. The genomic DNA was transferred overnight by capillary blotting with 10×SSC onto a Hybond-N+ (Amersham Pharmacia Biotech) membrane. The membrane was UV cross-linked and pre-hybridized in 50% formamide, 5×SSPE, 0.5% SDS, 5× Denhards, and Carrier DNA (2.5-3.5 mg/50 mL), for 4 hours at 42° C. The probe for hybridization was obtained by digesting the EGFP/Neo-diipMG+U6shRNA plasmid with EcoRI and Bam HI RE and labeling the resulting ˜320 bp U6shRNA cassette with the Random Primer Labeling system kit (Amersham Pharmacia Biotech). Following overnight hybridization at 42° C., the membrane was washed once at 37° C. with 6×SSPE, 0.1% SDS for 10 minutes, and twice with 2×SSPE, 0.1% SDS for 10 minutes.

### EXAMPLE 7

Long-term down-regulation of cell-surface expression of HLA class I gene on Jurkat T cells was obtained after transfecting the cells with a DNA plasmid expressing six copies of the U6 promoter and 705 stem-loop cassette and staining with PE-conjugated anti-HLA ABC. The cells were transfected and maintained in continuous culture for approximately 6 months (FIG. 4A).

Parental Jurkat cells also were transfected with DNA plasmid EGFP/diipMGNeo (no U6 promoter nor stem-loop cassette) and stained with PE-conjugated anti-HLA ABC (FIG. 4B).

Parental Jurkat cells also were transfected with DNA plasmid EGFP/diipMGNeo (no U6 promoter nor stem-loop cassette) and stained with PE-conjugated isotype control (FIG. 4C). The transfected cells were analyzed by flow cytometry for expression of EGFP and binding of PE-conjugated anti-HLA ABC. Dead cells were excluded from analysis by uptake of 1 mg/mL propidium iodide.

### EXAMPLE 8

Cell-surface expression of HLA class I gene was down-regulated on Jurkat T-cell clones transfected with a DNA plasmid expressing six copies of the U6 promoter and stem-loop cassette. The cells were plated at limiting dilution in 96-well round-bottom plates. Wells exhibiting growth were analyzed by flow cytometry for EGFP and binding of PE-conjugated anti-HLA ABC. Dead cells were excluded from analysis by uptake of 1 mg/mL propidium iodide.

### EXAMPLE 9

Cell-surface expression of HLA A3 and HLA B7 on T cells was down-regulated with a DNA plasmid expressing 0-10 copies of the U6 promoter and 705 stem-loop cassette. The cells were transiently transfected (FIG. 5A) or stably transfected (Jurkat cells resistant to cytocidal concentration of neomycin) (FIG. 5B). Jurkat cells were analyzed by multiparameter flow cytometry and electronically gated for EGFP expression. Percentage binding of either biotin-conjugated anti-HLA-A3 (One Lambda Corp.) or biotin-conjugated anti-HLA-B7/B27 (One Lambda Corp.), identified by binding of streptavidin-PE (PharMingen) also was measured relative to the binding of these same mAbs to untransfected (parental) Jurkat cells. Dead cells were excluded from analysis by uptake of 1 mg/mL propidium iodide.

### EXAMPLE 10

Cell-surface expression of HLA A2 was down-regulated on primary human HLA-A2⁺ T cells transfected with a DNA plasmid expressing 6 copies of the U6 promoter and stem-loop cassette. T cells were analyzed by flow cytometry for binding of PE-conjugated anti-HLA-A2 (Pharmingen). Dead cells were excluded from analysis by uptake of 1 mg/mL propidium iodide. The results are presented as the mean fluorescent intensity (MFI), which is a measure of the amount of expressed protein (FIG. 6A). This number was calculated using CellQuest (BD Sciences).

The expression of HLA class I as measured by PE-conjugated anti-HLA-ABC (PharMingen) on (I) parental T cells (derived from donor CA) that were not transfected (designated CA-OKT3) and (ii) hygromycin-resistant primary T cells (designated CA-MHC843+1) also was examined (FIG. 6F). The percent down-regulation (lower left box) is shown. The non-viral gene transfer of the DNA expression plasmid was achieved under the conditions described by Cooper, L J, et al., 2003. Briefly, peripheral blood mononuclear cells (PBMC) were stimulated with 30 ng/mL of anti-CD3 (OKT3, Ortho Biotech) and 3 days later the T cells were electroporated using the Eppendorf Electroporator with 5 mg of linearized DNA plasmid. Cytocidal concentrations of hygromycin (0.2 mg/mL) were added on day 5 of culture. T cells were maintained in RPMI 1640 (BioWhittaker, Walkersville, Md.) supplemented with 2 mM L-Glutamine (Irvine Scientific, Santa Ana, Calif.), 25 mM HEPES (Irvine Scientific), 100 U/mL penicillin, 0.1 mg/mL streptomycin (Irvine Scientific) and 10% heat-inactivated defined fetal calf serum (FCS) (Hyclone, Logan, Utah). Recombinant human IL-2 at 25 U/mL was added every 48 hours, beginning on day 1 of culture. 1×10⁶ T-cells were restimulated every 14 days by adding 30 ng/mL OKT3, 50×10⁶ irradiated PBMC and 10×10⁶ irradiated LCL. I1-2 and hygromycin were added as before.

### EXAMPLE 11

To generate HLA A2^{neg} T cells that could be eliminated in vivo by ganciclovir-mediated ablation (Bonini C., et al., 1997), HLA A2⁺ primary T cells were transfected with the HyTK-pMG plasmid, modified to express 6 copies of the HLA A-specific shRNA. Hygromycin-resistant T cells could be demonstrated to have down-regulated HLA A2-expression, relative to drug-resistant parental T-cell controls that do not express shRNA (FIG. 6A). As expected, there was only a small decrease in the binding of anti-HLA ABC to the T cells that had down-regulated HLA A2 expression, reflecting the fact that this mAb recognized epitope also present on HLA B and C molecules (FIG. 6A insert).

### EXAMPLE 12

In FIG. 6D, a Jurkat EGFP⁺ clone was incubated in serum-free media for 15 hours at 37° C. with the HIV peptide RLRPGGKKK [SEQ ID NO: 4] (derived from the p17 subregion of HIV gag) at 1 mg/mL. (Cooper, L. J., et al., 2003; Lewinsohn, D. A., et al., 2002) This concentration of peptide resulted in maximal CTL-mediated specific lysis of non-transfected HLA A3⁺ Jurkat parental cells using a standard 4-hour chromium release assay (CRA) (data not shown). After washing to remove unbound peptide, the RLRPGGKKK [SEQ ID NO: 4]-specific HLA A3⁺ CD8⁺ T-cell clone 28A2-15 (Lewinsohn, D. A., et al., 2002) was added at an effector:target (E:T) ratio of 5:1 and the cells were co-cultured for 4 hours at 37 ° C. This E:T ratio results in maximal lysis, assessed by CRA of non-transfected Jurkat parental cells that have been incubated with 1 mg/mL peptide (data not shown). The cells were then stained with biotin-conjugated mAb specific for HLA A3, followed by PE-conjugated streptavidin and analyzed by flow cytometry for the presence of PE staining on the remaining EGFP⁺ cells that excluded 1 μg/mL PI.

### EXAMPLE 13

EGFP expression was down-regulated in neomycin-resistant primary T cells stably transfected with 8 copies of the U6 promoter and 705 stem-loop cassette (FIG. 7). T cells were analyzed by multiparameter flow cytometry for expression of EGFP (x-axis) and binding of PE-conjugated anti-HLA-ABC (Pharmingen) or PE-conjugated isotype- and concentration-matched mAb control. Dead cells were excluded from analysis by uptake of 1 mg/mL propidium iodide. The percentage of T cells expressing EGFP is given for each plot. The MFI for the binding of the PE-conjugated anti-HAL-ABC is given for each plot.

### EXAMPLE 14

RNA and cDNA preparation. Total RNA was extracted using Rneasy Mini Kit (QIAGEN Inc., Valencia, Calif.) according to the manufacturer's instructions. cDNA was made by reverse transcription (RT) for 1 hour at 42° C. from 3 μg of RNA, 67 pmol of oligo dT, 0.2 mM dNTP, 0.3 μL of Rnase inhibitor, 0.1 M DTT, and 1 μL of reverse transcriptase (200 U, Superscript II, Invitrogen, Carlsbad, Calif.) in a 30 μL reaction. The reaction was then heat inactivated at 95° C. for 5 minutes and the mixture was used directly for PCR.

### EXAMPLE 15

RT-PCR. To simultaneously determine the relative HLA-A and HLA-B mRNA levels, first-strand cDNA from Jurkat cells was used to PCR-amplify (one cycle of 95° C. for 9 min, 20 cycles of 94° C. for 30s; 58° C. for 20s; 72° C. for 30 s, and one cycle of 72° C. for 10 min) both the 250 bp replicon using HLA A-specific primers and a 150 bp replicon using HLA B-specific primers, which were resolved by electrophoresis in a 1.5% agarose gel developed with ethidium bromide (ETBr). The HLA A-, B-, C-specific 5′ primer was designated Q-ABC5 (5′-GCTGTGGTGGTGCCTTCTGG-3′ [SEQ ID NO: 5]), the HLA A-specific 3′ primer was designated A013 (5′-CCTGGGCACTGTCACTGCTT-3′ [SEQ ID NO: 6]) and HLA B-specific 3′ primer was designated B013 (5′-CCTGGGCACTGTCACTGCTT-3′ [SEQ ID NO: 6]) (Johnson, D. R., et al., 2003). The relative binding of these primers to an HLA class I sequence is shown (FIG. 1G). The PCR cycles were pre-determined to be in the linear range. Quantification was undertaken using LabWorks™ Image Acquisition and Analysis Software, version 4.0.0.8 (UVP Biolmaging Systems, Upland Calif.) normalizing for the ETBr in a 1-kb ladder (NEB; MA,CA). Independent PCR reactions, normalizing against GAPDH in each sample, ensured that the two sets of primers used in each PCR reaction did not compete with other. Since the primers span an intron, genomic contamination was excluded by the absence of high molecular weight PCR product.

The publications and other materials cited herein to illuminate the background of the invention and to provide additional details respecting the practice of the invention are incorporated herein by reference to the same extent as if they were individually indicated to be incorporated by reference.

While the invention has been disclosed by reference to the details of preferred embodiments of the invention, it is to be understood that the disclosure is intended in an illustrative rather than a limiting sense, as it is contemplated that modifications will readily occur to those skilled in the art, within the spirit of the invention and the scope of the appended claims.

### REFERENCES

1. Adams, E. J. and Parham, P. (2001). Genomic analysis of common chimpanzee major histocompatibility complex class I genes. Immunogenetics 53:200-208.
2. York, I. A., Roop C., Andrews D. W., Riddell S. R., Graham F. L., and Johnson D. C. (1994). A cytosolic herpes simplex virus protein inhibits antigen presentation to CD8+t lymphocytes. Cell 77:525-535.
3. Lorenzo, M. E., Ploegh, H. L., and Tirabassi R. S. (2001). Viral immune evasion strategies and the underlying cell biology. Semin Immunol. 13:1-9.
4. Berger, C. et al. (2000). Expression of herpes simplex virus ICP47 and human cytomegalovirus US11 prevents recognition of transgene products by CD8(+) cytotoxic T lymphocytes. J Virol. 74:4465-4473.
5. Barel, M. T., Pizatto N., van Leeuwen D., Bouteiller P. L., Wiertz E. J., and Lenfant F. Amino acid composition of alpha1/alpha2 domains and cytoplasmic tail of MHC class I molecules determine their susceptibility to human cytomeglovirus US11-mediated down-regulation. (2003) Eur. J Immunol. 33:1707-1716.
6. Lee, N. S. et al. (2002). Expression of small interfering RNAs targeted against HIV-1 rev transcripts in human cells. Nat Biotechnol. 20:500-505.
7. Brummelkamp, T. R., R. Bernards, and R. Agami, A system for stable expression of short interfering RNAs in mammalian cells. Science, 2002. 296(5567): p. 550-3.
8. Hakem, R. et al. (1989). IFN-mediated differential regulation of the expression of HLA-B7 and HLA-A3 class I genes. J Immunol. 142:297-305.
9. Sukulev, U., Joo, M., Vturina, I., Tsomides, T. J., and Eisen, H. N. (1996). Evidence that a single peptide-MHC complex on a target cell can elicit a cytolytic T cell response. Immunity. 4:565-571.
10. Cooper, L. J. et al. (2003). Enhancing siRNA-effects in T Cells. Blood. 102:2757 (2003) [abstract].
11. Johnson, D. R. (2003). Locus-specific constitutive and cytokine-induced HLA class I gene expression. J Immunol. 170:1894-1902.
12. Elbashir, S. M., et al., Duplexes of 21-nucleotide RNAs mediate RNA interference in cultured mammalian cells. Nature, 2001. 411(6836): p. 494- 8.
13. Harborth, J., et al., J. Cell Sci. 114:4557-4565, 2001.
14. Yu, J. Y., S. L. DeRuiter, and D. L. Turner, RNA interference by expression of short-interfering RNAs and hairpin RNAs in mammalian cells. Proc Natl Acad Sci USA 2002. 99(9): p. 6047-52.
15. Miyagishi, M. and K. Taira, U6 promoter driven siRNAs with four uridine 3′ overhangs efficiently suppress targeted gene expression in mammalian cells. Nat Biotechnol, 2002. 20(5): p. 497-500.
16. Paul, C. P., et al., Effective expression of small interfering RNA in human cells. Nat Biotechnol, 2002. 20(5): p. 505-8.
17. Ketting, R. F., et al., Dicer functions of RNA interference and in synthesis of small RNA involved in developmental timing in C. elegans. Genes Dev, 2001. 15(20): p. 2654-9.
18. Parham, P., Genomic organization of the MHC: structure, origin and function. Immunol.

Rev 167:5-379, 1999.
19. Paule, M. R., et al., Nucleic Acids Res. 28:1283-1298, 2000.
20. Holen T., et al., “Positional effects of short interfering RNAs targeting the human coagulation trigger Tissue Factor,” Nucleic Acids Res. 30(8):1757-66, Apr. 15, 2002.
21. Sohail, M., et al., “A simple and cost-effective method for producing small interfering RNAs with high efficacy,” Nucleic Acids Res. 31(7):E38, Apr. 1, 2003.
22. Barton, G. M., et al., “Retroviral delivery of small interfering RNA into primary cells,” Proc. Natl Acad. Sci USA 99(23):14943-5, 2002.
23. Cooper, L. J., et al., “T-cell clones can be rendered specific for CD19: toward the selective augmentation of the graft-versus-B-lineage leukemia effect,” Blood 101:1637-44, 2003.
24. Lupton, S. D., et al., “Dominant positive and negative selection using a hygromycin phosphotransferase-thymidine kinase fusion gene,” Mol. Cell. Biol. 11:3374-3378, 1991.
25. Castanotto et al., 2002, RNA 8:1454-60.
26. Bonini, C., et al., “HSV-TK gene transfer into donor lymphocytes for control of allogeneic graft-versus-leukemia,” Science 276:1719-1724, 1997.
27. Lewinsohn, D. A., et al., “HIV-1 Vpr does not inhibit CTL-mediated apoptosis of HIV-1 infected cells,” Virology 294:13-21, 2002.
28. Morris, K. V., et al., “Small Interfering RNA-Induced Transcriptional Gene Silencing in Human Cells,” Science 305 (5688): 1289-92, 2004.
29. Kawasaki, H., et al., “Induction of DNA methylation and gene silencing by short interfering RNAs in human cells,” Nature 431 (7005): 211-17, 2004.

1. A method for down-regulating expression of a target gene in a mammalian cell, comprising:
introducing into the mammalian cell a RNAi-expressing concatamer in the form of an expression vector comprising a plurality of promoter-RNAi expression cassettes, wherein RNAi is expressed and initiates RNA interference of expression of the target gene, thereby down-regulating expression of the target gene.

2. The method of claim 1, wherein the target gene is a HLA class I gene.

3. The method of claim 2, wherein the expression vector comprises 4 to 6 promoter-RNAi expression cassettes.

4. The method of claim 2, wherein the expression vector comprises 6 to 8 promoter-RNAi expression cassettes.

5. The method of claim 2, wherein the expression vector comprises 6 promoter-RNAi expression cassettes.

6. The method of any one of claim 2, wherein the promoter is a U6 Pol III promoter.

7. The method of claim 6, wherein one or more of the promoter-RNAi expression cassettes comprise a stem-loop DNA sequence having the sequence
[SEQ ID NO: 1]
5′GGAGATCACACTGACCTGGCAtttgtgtagTGCCAGGTCAGTG
TGATCTCC3′.

8. The method of claim 6, wherein one or more of the promoter-RNAi expression cassettes comprise a stem-loop DNA sequence having the sequence
[SEQ ID NO: 3]
5′CACCTGCCATGTGCAGCATGAtttgtgtagTCATGCTGCACAT
GGCAGGTG3′.

9. A method for amplifying expression of RNAi in a mammalian cell, comprising:
introducing into the mammalian cell a RNAi-expressing concatamer in the form of an expression vector comprising a plurality of promoter-RNAi expression cassettes which express RNAi.

10. An RNAi-expressing concatamer in the form of an expression vector comprising a plurality of promoter-RNAi expression cassettes.

11. The RNAi-expressing concatamer of claim 10, wherein the plurality of promoter-RNAi expression cassettes are arranged in tandem.

12. The RNAi-expressing concatamer of claim 10, wherein the promoter is a U6 Pol III promoter.

13. A method for disrupting antigen presentation by a cell by down-regulating HLA gene expression in the cell with RNAi, comprising:
introducing into the cell one or more expression cassettes encoding an RNAi molecule corresponding to a gene encoding a MHC class I gene, wherein the RNAi molecule is expressed and initiates RNA interference of expression of the MHC gene, thereby down-regulating expression of the MHC gene and disrupting antigen presentation.

14. The method of claim 13, wherein the cell is a human T cell and the MHC gene encodes HLA ABC.

15. The method of claim 14, wherein the T cell contains 6-8 copies of the RNAi encoding cassette in a single DNA plasmid.

16. The method of claim 13, wherein the cell is protected from lysis by T cells.
